(19) United States
(12) Patent Application Publication
(10) Pub. No.: US 2003/0153776 A1
(43) Pub. Date: Aug. 14, 2003

(54) Process for obtaining betulin

(75) Inventors: Markus Sauter, Gensingen (DE); Carsten Bender, Ummendorf (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/356,295

(22) Filed: Jan. 31, 2003

Related U.S. Application Data
(60) Provisional application No. 60/386,266, filed on Jun. 6, 2002.

(30) Foreign Application Priority Data
Feb. 2, 2002 (DE) 10204278.0

(51) Int. Cl. C07J 53/00

(52) U.S. Cl. 552/511

(57) ABSTRACT

The invention relates to an improved process for obtaining pure crystalline betulin which is substantially free from betulinic acid, from an extract of birch bark.

### RELATED APPLICATIONS

Benefit of U.S. Provisional Application Serial No. 60/386,266, filed on Jun. 6, 2002 is hereby claimed, and said Application is herein incorporated by reference.

### DESCRIPTION

The invention relates to an improved process for obtaining pure crystalline betulin which is substantially free from betulinic acid, from an extract of birch cortex.

### BACKGROUND OF THE INVENTION

Betulin is 3β,28-dihydroxy-lup-20(29)-ene of formula

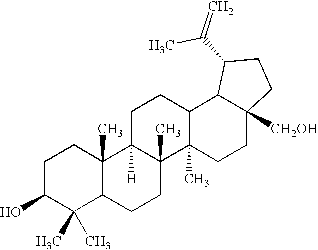

It is known that betulin is used as an anti-ageing agent in skin creams. Consequently, betulin is in great demand.

Russian Patent RU 2138508 proposes treating a birch bark extract with concentrated sodium hydroxide solution and separating off the sodium salts of fatty acids and triterpene acids thus obtained in solid form.

However, this process is unsuitable for the industrial recovery of large amounts of betulin as the separation of these salts by decanting or centrifuging, for example, is very time-consuming and leads to reduced space-time yields.

The problem of the present invention was therefore to provide a process which makes it possible to produce betulin with a high degree of purity in high space-time yields.

### DETAILED DESCRIPTION OF THE INVENTION

Surprisingly it has now been found that highly pure crystalline betulin can be obtained from an extract of birch bark if this extract is extracted with a dilute aqueous base and the aqueous phase is separated off.

The invention thus relates to a process for obtaining highly pure crystalline betulin by extracting birch bark with a high-boiling water-immiscible solvent, wherein the extract obtained or the residue thereof dissolved in another water-immiscible solvent is extracted with a dilute aqueous base and the aqueous phase is separated off.

The word “betulin” as used above and hereinafter includes both betulin as such and the hydrates and solvates thereof, preferably betulin, which is solvated with 0.2 to 1.0, particularly 0.5 equivalents of an alcohol, and most particularly one equivalent of ethanol.

The betulin prepared by the process according to the invention is substantially free from betulinic acid, as a rule it contains no betulinic acid or betulinic acid in an amount which cannot be detected by conventional methods of analysis such as HPLC, for example.

Preferably, the betulin prepared by the process according to the invention contains less than 1% by weight, preferably less than 0.1% by weight, and most preferably less than 10 ppm betulinic acid.

Suitable high-boiling water-immiscible solvents generally include aromatic hydrocarbons with a boiling point above 65° C., particularly above 80° C. or mixtures thereof, preferably aromatic hydrocarbons with 6 to 9 carbon atoms, and most preferably toluene or xylene.

Suitable dilute aqueous bases are generally inorganic bases which are capable of converting an organic carboxylic acid into the corresponding carboxylate, preferably alkali metal or alkaline earth metal hydroxides, such as for example lithium hydroxide, sodium hydroxide, potassium hydroxide or calcium hydroxide or alkali metal or alkaline earth metal carbonates, such as for example sodium carbonate or potassium carbonate, particularly sodium hydroxide or potassium carbonate, while potassium carbonate is most particularly preferred.

The dilution of these bases is generally performed so that the salts of the fatty acids and triterpene acids formed are completely dissolved in the aqueous phase. Particularly preferred are aqueous solutions containing 1 to 10% by weight, and most preferably 2 to 8% by weight of inorganic base. In the event that hereinabove or herein-below the relation of two compounds is indicated in the form of “x to y fold amount of the first compound”, wherein x and y represent the lower and upper limit of said amount, this indication relates to “x to y” parts per weight of said first compound with respect to 1 part per weight of the second compound.

Other suitable water-immiscible solvents are generally aromatic hydrocarbons such as for example toluene or xylene, or aliphatic or cycloaliphatic ethers, preferably aliphatic ethers such as for example methyl-tert-butylether (MTB) or diisopropylether.

Suitable alcohols for the recrystallisation of the crude betulin are generally aliphatic alcohols with 1 to 4 carbon atoms, particularly methanol, ethanol or isopropanol or mixtures thereof, most preferably ethanol.

Preferred embodiments of the invention are:

(A) processes in which the following steps are carried out successively:

(i) heating the ground-up birch bark in a high-boiling, water-immiscible solvent;

(ii) filtering the extract;

(iii) optionally concentrating the extract thus obtained, precipitating it with a nonpolar solvent and dissolving the residue in a water-immiscible solvent;

(iv) extracting the solution obtained in step (ii) or (iii) with a dilute aqueous base;

(v) eliminating the aqueous phase and concentrating the organic phase;

(vi) optionally precipitating with a nonpolar solvent;

(vii) heating, clarifying and crystallising the product of step v or vi it from an alcohol; and

(viii) separating the betulin from the alcoholic phase.

As a rule, aliphatic or cycloaliphatic hydrocarbons or mixtures thereof, such as for example n-pentane, n-hexane, petroleum ether with a boiling point in the range from 50 to 100° C., cyclohexane or methylcyclohexane are suitable as nonpolar solvents for precipitating the betulin in step (iii) or (vi).

Also preferred are:

(B) processes in which the ground birch bark is heated one to three times in each case with 1.5 to 3 times the amount of toluene for 0.5 to 10.0 hours and the extract obtained is filtered at elevated temperature.

(C) processes in which the extract from step (ii) or (iii) is extracted one to five times with a 1 to 10%, preferably about 5% aqueous sodium or potassium carbonate solution or a 0.1 to 1.0 N, preferably about 0.5 N aqueous sodium or potassium hydroxide solution.

(D) processes in which in step (iii) the extract is concentrated under reduced pressure, the residue is washed with petroleum ether and dried, and the crystals remaining are dissolved in toluene or an ether, preferably MTB.

(E) processes in which the residue in step (iii) is dissolved in 10 to 100 times the quantity of an ether, particularly MTB.

(F) processes in which the alcoholic phase in step (vii) is heated to temperatures of 50 to 120° C., particularly 60 to 95° C., optionally in the presence of activated charcoal and filtered while hot.

(G) process in which in step (viii) the betulin crystallises out as the alcoholic phase cools.

A most particularly preferred embodiment of the process according to the invention comprises the following steps (i) to (viii):

(i)/(ii) The coarsely ground birch bark is suspended in 2 to 5 times, preferably 3 to 4 times the amount of toluene and heated for 1 to 10 hours, particularly about 4 hours, preferably at 80° C. After the plant residues have been filtered they are heated again, preferably to boiling, with 2 to 5 times, preferably 3 to 4 times the amount of toluene for 1 to 5 hours, most preferably about 2 hours, and filtered once more.

(iii) The combined extracts are optionally concentrated by evaporation in vacuo at 40 to 70° C. The concentrated extract is taken up with petroleum ether or methylcyclohexane, preferably petroleum ether (60-95° C.), the precipitate formed is suction filtered and washed with petroleum ether or methylcyclohexane. The residue is preferably dried in vacuo at 40 to 70° C., particularly at about 50° C. In this way, crude betulin is obtained (about 2.0 to 4.0%, based on birch bark). This is dissolved in 10 to 100 times, particularly 20 to 50 times the amount of MTB or in 100 to 300 times, most preferably 150 to 250 times the amount of toluene.

(iv) The toluene extract obtained from (ii) or the MTB or toluene solution from (iii) is extracted once to three times with a 2 to 10% potassium carbonate solution or a 0.2 to 0.8 normal sodium hydroxide solution.

(v) The aqueous phase is removed and the organic phase evaporated down in vacuo.

(vi) The crude betulin obtained may optionally be precipitated by the addition of petroleum ether or methylcyclohexane.

(vii) The betulin obtained according to (v) or (vi) is refluxed in the presence of activated charcoal in 10 to 50 times the amount of ethanol and filtered while hot. As the mother liquor cools the pure betulin begins to crystallise out.

(viii) The mixture is cooled to about 5° C., and the crystals obtained are suction filtered. After drying in a vacuum cupboard at about 90° C., highly pure, crystalline betulin is obtained, which is solvated with 0.5 mol ethanol in the form of white crystals with a melting point of 257-259° C.

The process according to the invention makes it possible to prepare pure betulin with a content of betulinic acid far below 1%. The process can easily be carried out on an industrial scale as it can be done using cheap reagents and without any time-consuming separation steps, and gives good to very good yields.

The following Examples serve to illustrate the process for obtaining betulin which is carried out by way of example. They are intended solely as possible procedures provided as an illustration, without restricting the invention to their contents.

### EXAMPLE 1

750 g of coarsely ground birch bark are decocted for 4 hours at 80° C. with 2.5 l of toluene. After the plant residues have been filtered the mixture is again decocted with 2.0 l of toluene and filtered off. The combined mother liquors are washed three times with one litre of a 5% aqueous potassium carbonate solution. After phase separation the organic phase is evaporated down under reduced pressure. 30.0 g (4% based on birch bark) of crude betulin are obtained as almost white crystals. No betulinic acid can be detected by DC (against a comparison).

### EXAMPLE 2

2.5 kg of coarsely ground birch bark are stirred in 4.5 l of toluene at 70° C. for 2 hours. The toluene is decanted and the bark is again extracted for 2 hours with 4.0 l of toluene at 70° C. and then filtered off. The combined filtrates are evaporated almost to dryness in vacuo at 50° C. and combined with 500 ml of petroleum ether (60-95° C.) and stirred for 2 hours at ambient temperature. The resulting crystal slurry is suction filtered and the crystals are washed with 500 ml of petroleum ether (60-95° C.) and dried at 50° C. in a vacuum cupboard. 45 g (2.8% yield based on birch bark) of crude betulin are obtained with an HPLC content of 70.2% and a betulinic acid content of 6.1%.

### EXAMPLE 3

39 g of betulin from Example 2 are dissolved in 8000 ml of toluene, extracted once with 0.5 normal sodium hydroxide solution and washed with 2000 ml of water. The organic phase is separated off and evaporated down in vacuo. The residue (29 g) is recrystallised from 700 ml of ethanol (filtration with pressure filter). After drying overnight at 50° C., 14.6 g of betulin are obtained in a 37% yield with a content of 89.9%. No betulinic acid can be detected by HPLC. ¹H-NMR: crystal contains about 0.5 mol-% ethanol.

The mother liquor yields a further 3.7 g of betulin by distillative working up (NMR: slightly contaminated).

Recrystallisation of Crude Betulin

50 g of crude betulin (prepared according to Example 3) and 15 g of activated charcoal are refluxed in 1.00 l of ethanol and filtered while hot. As the mother liquor cools, the betulin begins to crystallise. It is cooled to about 5° C., and the crystals obtained are suction filtered. After drying in the vacuum cupboard at about 90° C., 27.9 g (56% yield) of pure betulin is obtained in the form of white crystals, with a melting point of 257-259° C., a rotational value [α]_{D} of +19.4 (c=2 in pyridine), and a content of 95.8% (HPLC against an external standard).

We claim:

1. A process for obtaining highly pure crystalline betulin by extraction from birch bark with a high-boiling, water-immiscible solvent, characterised in that the extract obtained or the residue thereof dissolved in a water-immiscible solvent is extracted with a dilute aqueous base and the aqueous phase is separated off.

2. Process according to claim 1, characterised in that the following steps are carried out successively:
(i) heating the coarsely ground-up birch bark in a high-boiling, water-immiscible solvent;
(ii) filtering the extract;
(iii) optionally concentrating the extract thus obtained and taking up the residue in a water-immiscible solvent;
(iv) extracting the solution thus obtained with a dilute aqueous base;
(v) eliminating the aqueous phase and concentrating the organic phase;
(vi) optionally precipitating with a nonpolar solvent;
(vii) heating, clarifying and crystallising it from an alcohol; and
(viii) separating the betulin from the alcoholic phase.

3. Process according to claim 1, wherein the coarsely ground birch bark is heated once or three times in each case with 1.5 to 3 times the amount of toluene for 0.5 to 10.0 hours and the extract obtained is filtered at elevated temperature.

4. Process according to claim 2, wherein the extract from step (ii) or (iii) is extracted one to five times with a solution selected from the list consisting of 1 to 10% aqueous sodium or potassium carbonate solution or a 0.1 to 1.0 N aqueous sodium or potassium hydroxide solution.

5. Process according to claim 2, wherein the extract of step (iii) concentrated under reduced pressure, the residue is taken up in petroleum ether, washed and dried, and the crystals remaining are dissolved in toluene or an ether.

6. Process according to claim 2, wherein the residue in step (iii) is dissolved in 10 to 100 times the amount of an ether.

7. Process according to claim 2, wherein the alcoholic phase in step (vii) is heated to temperatures of 50 to 120° C., optionally in the presence of activated charcoal and filtered while hot.

8. Process according to claim 1, wherein in step (viii) the betulin crystallises out as the alcoholic phase cools.
